(19) United States
(12) Patent Application Publication
(10) Pub. No.: US 2004/0151756 A1
(43) Pub. Date: Aug. 5, 2004

(54) Edible low density high surface area drug vehicle, method of manufacturing low density high surface area drug vehicle

(76) Inventors: Anthony P. Richards, Gary, IN (US); Thomas J. Hinkemeyer, Gages Lake, IL (US); Robert F. Boutin, Hinsdale, IL (US)

Correspondence Address: Michael R. McKenna, Suite 3800, 500 West Madison, Chicago, IL, 60661, US

(21) Appl. No.: 10/357,811

(22) Filed: Feb. 4, 2003

(51) Int. Cl. A61K 47/00

(52) U.S. Cl. 424/439

(57) ABSTRACT

An edible foam carrier is provided comprising aerated stabilizers adapted to receive a medicament. Also provided is a method for producing an edible foam carrier, the method comprising aerating a water-soluble stabilizer to create a three-dimensional structure, and confining medicaments to the structure. The structure can be homogenous or the result of multi-extrusion processes whereby a core and a sleeve are coextruded to define a single construct.

### BACKGROUND OF THE INVENTION

1. Field of the Invention

This invention relates to an edible foam carrier and a method for producing an edible foam carrier, and more particularly, this invention relates to a method for producing palatable medicament carriers to facilitate almost immediate medicament dispersion and/or dissolution into the blood stream via the oral cavity and its transmucal membranes.

2. Background of the Invention

Research continues for the ideal mode to administer medicaments that is convenient for both medical personnel and patients. The search for this ideal mode is complicated by the characteristics of certain drugs; i.e., sensitivity to processing heat, water and moisture, and other ingredients.

Particularly in oral suspensions, efforts are ongoing to provide delivery vehicles to facilitate immediate dispersal of actives without the need for water or coercion.

Previous efforts centered on chewable forms and lozenges to effect dispersal. A drawback to these modes is that fast release of medicaments is not possible. Another drawback is that the relatively dense construct of chewables and lozenges restricts actives loading to low dosages.

Efforts have been made to develop high-volume oral dispersants. Generally, these efforts have been lacking.

U.S. Pat. No. 6,090,401 awarded to Gowan et al on Jul. 18, 2000 discloses a stable foam composition for use as a carrier in oral medicines. However, the '401 formulation is too light weight to provide adequate dosages in many treatment regimens.

U.S. Pat. Nos. 4,639,367 and 4,752,465 awarded to Mackles on Jan. 27, 1987 and Jun. 21, 1988 respectively disclose an edible foam having a whipped cream consistency. The '367 formulation suspends up to 50 percent by weight of solid particles for administration. Medicaments utilized in the '465 formulation are oily medicinal agents and thus serve to stymie oxidation of such agents. Formulations from both the '367 and '465 patents are semi-solid and not suitable for forming into discrete medicament-delivery packets.

U.S. Pat. Nos. 5,369,131 awarded to Poli et al on Nov. 29, 1994 and U.S. Pat. No. 5,458,884 awarded to Britton et al on Oct. 17, 1995 disclose liquid pharmaceutical delivery systems for topical use. Generally, the systems facilitate the formation of a medicament-loaded a film over the area to be treated or lubricated for protracted time periods, such areas often being mucosa-lined.

U.S. Pat. No. 4,780,309 awarded to Geria et al on Oct. 25, 1988 discloses a foam for dispensing unpleasant tasting oil. Each teaspoon of the foam utilizes an inorganic complexing agent to sequester from about 2 to 4 grams of oil.

A need exists in the art for an oral suspension for medicaments that facilitates rapid, yet pleasant dispersal of the therapeutic material. The suspension should be manufactured with inexpensive ingredients and should favor heavy loading of the therapeutic material, whether that material is of a soluble or insoluble nature, and whether the material initially is in liquid or solid phase.

### SUMMARY OF THE INVENTION

It is an object of the invention to provide a method for producing orally dispersed medicament carriers that overcomes many of the disadvantages of the prior art.

Another object of the present invention is to provide an economical medicament carrier. A feature of the invention is its ability to sequester up to 33 percent of its own weight in medicament, whether that medicament is soluble or insoluble. An advantage of the invention is that the high dosing feature allows for oral deployment of therapeutic dosages of the medicament.

Still another object of the present invention is producing an extrudable, foam-based medicament carrier at low temperatures and low water activity levels. A feature of the invented carrier is that it is capable of manipulation immediately upon extrusion and before dehydration to provide localized concentrations of cellular structures adapted to sequester predetermined volumes of medicaments. An advantage of the invented carrier is that extruded objects “cure” or otherwise set-up to form a smooth surface over the cellular structures, thereby assuring even-moisture uptake, uniform release of active ingredients, and low friability. Another advantage is that the resulting carrier presents a stable drug delivery system for heat-sensitive actives. Aside from being extrudable, the invented medicament foam phase can be deposited directly into molds or directly into sales packaging. In addition, formulations for facilitating the formation of the invented foam medicaments into slabs are presented herein.

Yet another object of the present invention is providing an aerosolized foam carrier of active chemicals for administration by placing same in certain physiologic fenestra. A feature of the invention is that the carrier can contain effervescing agents. An advantage of the invention is that the carrier, with or without effervescing agents, facilitates nearly instantaneous delivery of the active chemicals once the carrier contacts moisture endemic of the particular fenestra (i.e., mouth, ear, nostril, rectum, vagina, etc). Another advantage is that the effervescing effect is a physical cue to the patient that dispensing of the medicament is occurring.

Briefly, the invention provides an edible foam carrier comprising aerated protein adapted to receive a medicament.

The invention also provides a method for producing an edible foam carrier, the method comprising aerating a water-soluble protein, or other suitable stabilizer (i.e., starches, gums) to create a three-dimensional structure, and temporarily sequestering medicament to the structure.

### BRIEF DESCRIPTION OF THE DRAWING

The invention together with the above and other objects and advantages will be best understood from the following detailed description of the preferred embodiment of the invention shown in the accompanying drawing, wherein:

FIG. 1 is a schematic diagram of an extrusion process for producing discrete constructs of the invented high volume foam carrier, in accordance with features of the present invention; and

FIG. 2 is a schematic diagram of a co-extrusion process, in accordance with features of the present invention.

### DETAILED DESCRIPTION OF THE INVENTION

The present invention provides a dosage form, such as a foam matrix, whereby the matrix is adapted to carry active materials for nearly instantaneous release in the oral cavity or in fenestra having high moisture content. The ability of this foam construct to be processed at temperatures up to approximately 115° C. (240° F.) allows the incorporation of a broad base of heat sensitive active ingredients.

The invented foam can accommodate delivery of several different types of actives, including, but not limited to, anti-microbial agents, non-steroidal drugs, anti-tussives, decongestants, anti-histamines, expectorants, anti-diarrheals, H₂-antagonists, proton pump inhibitors, CNS depressants, CNS stimulants, CNS modifiers, anti-parkinsonism drugs, narcotic analgesics, analgesic-antipyretics, and psychophannocological drugs. Given the typical size of lozenges (approximately 1-10 cc), specific species and dosages of the above-identified genus of actives to be accommodated by the invented vehicle include the following:

| Pharmaceutical Active Agent | Preferred Dose (mg) |
|---|---|
| Chlorpheniramine Maleate | 4 |
| Brompheniramine Maleate | 2 |
| Dexchlorpheniramine | 2 |
| Dexbrompheniramine | 2 |
| Triprolidine Hydrochloride | 2.5 |
| Acrivastine | 8 |
| Azatadine Maleate | 1 |
| Loratidine | 10 |
| Phenylophrine Hydrochloride | 10 |
| Dextromethorphan Hydrobromide | 10-30 |
| Ketoprofen | 12.5-25 |
| Sumatriptan Succinate | 35-70 |
| Zolmitriptan | 2.5 |
| Loperamide | 2 |
| Famotidine | 10 |
| Nicotine | 2 |
| Diphenhydramine Hydrochloride | 12.5-25 |
| Pseudoephendrine Hydrochloride | 30 |

It should be noted that the above list is merely illustrative, and not exhaustive of the type or genus of active ingredients the instant invention can accommodate.

The method of manufacture of the invented foam construct yields a phase consistency to facilitate extrusion, sheeting, or simple deposition into final packaging materials.

Foam Carrier

Constituent Detail

A myriad of biological material can be utilized as the main foam constituent of the invented carrier. Either or both lyophilized and solubilized proteins are suitable, and are generally present in the entire construct at a weight percent of between approximately 0.1 and 10.

Suitable aerating agents include, but are not limited to, albumen, polymer binders, gums, starches, hydrocolloids, carboxymethyl cellulose, alginates, gelatins, and combinations thereof.

As to albumens, structures having the following general formula are suitable:

C₇₂H₁₁₂N₁₈O₂₂S

including those proteins found in white of egg, blood, lymph, chyle and many other animal and vegetable tissues.

Generally, the protein is combined with an anionic surfactant to enhance miscibility with saliva. Combination ratios for protein and surfactant range from between 1:100 and 1:5 on a mass basis. Exemplary surfactants are ionic surfactants generally, including but are not limited to, sodium lauryl sulphate, sodium laureth sulfate, ammonium laureth sulfate, sodium tridecyl ether sulfate, and combinations thereof.

The protein can be used alone or else admixed with buffers so as to confer stability to the foam construct in pH-variable situations. The invented system and method are viable within a wide pH range, from the very acidic to the very alkaline. A preferable pH range is between approximately 5 and 9. A more preferable range is between 5.5 to 8.5, with the later pH value the result of incorporating calcium carbonate as an active constituent.

The addition of the buffers allows the foam to maintain its integrity, and particularly confers additional wet-foam stability in a myriad of physiological pH ranges, and more generally within a pH range of between 4 and 10. Suitable buffers include solubilized protein such as solubilized milk protein, soy-based whipping proteins, salts (such as phosphates, bicarbonates, citrates, tartrates, and gluconates), and trace elements (such as calcium, magnesium, copper, iron, sodium and potassium), and combinations thereof.

Bulking agents are utilized to confer volume to the foam construct, Suitable volume enhancing agents include, but are not limited to, sucrose, 6-0-A-D-Glucopyranosyl-D-fructose, clay, calcium carbonate, talc, magnesium silicate, dextrose, lactose, polyols, various carbohydrate sweeteners (including but not limited to various grades of corn syrups, invert sugar, fructose, and maltose) and combinations thereof. Suitable stabilizing agents include, but are not limited to, gum arabic, corn starch, potato starch, dextrins, and combinations thereof.

Leavening agents are often added to the foam-construct liquor to confer added surface area per unit volume of the foam. Indeed, foam densities can vary from 0.05 gm/cc to 0.9 gm/cc, depending on loading of these agents. In general, the greater mass fraction of the agent produces a lower density product. Suitable leavening agents include, but are not limited to, dicalcium phosphate dihydrate, and sodium bicarbonate, yeast, ammonium bicarbonate, and sodium aluminum phosphates.

Surfactants and other surface wetting agents, such as polysorbate 60 (HLB=14.9 and sorbitan monosterate (HLB=4.7) are utilized to improve whippability, dispersion and dissolution. The weight percent of these two components to the entire construct is empirically derived, depending on the miscibility characteristics desired. An examplary weight percent is approximately 0.2.

Effervescent agents also can be added, suitable agents being sodium bicarbonate, and citric acid. Care is taken to maintain a quantity of unreacted effervescent material after the drying process so that an effervescent effect is obtained upon contact with physiological fluids. This may require the pretreatment of the bicarbonate with heat and or fats/polymers to increase its stability. Such pretreatment may include mild thermal exposure, sufficient to convert the outer layers of the bicarbonate granule to carbonate according to the following reaction process: 2NaHCO₃→Na₂CO₃+CO₂+H₂0. Time release of effervescence also can be obtained by encapsulation of bicarbonate granules with water insoluble coatings such as fats or other edible hydrophobic residues.

The above-enumerated constituents also can be utilized as texture modifiers, when present in concentrations such as those presented infra, in the examples. Also, taste-masking agents, widely commercially available, can be utilized depending on the palatability of the active com-pounds to be delivered by the invented carrier.

Before aeration, but after the above ingredients are homogeneously mixed, the active component (such as medicaments) is added to the foam carrier, also via homogenous mixing.

Aeration

Detail

Generally, the foam carrier is produced by aerating or otherwise agitating the foam-forming construct, either atmospherically or under pressure so as to inject air, nitrogen, helium, carbon dioxide, nitrous oxide, or some other relatively inert gas-phase component into the mixture. Such agitation or other type of mechanical action denatures the proteins comprising part of the foam-liquor. This denaturization leads to coagulation, resulting in stiffening of the foam and stabilization of air bubbles entrained in the mass. Care should be taken to not over agitate the mixture. Otherwise, this would lead to a nearly complete denaturing of the protein base and excessive coagulation of the liquor.

The extrusion characteristics of the final construct come from the non-flowing stiff foam having a very fine, tightly packed air cell structure of a density of between 0.05 and 0.9 gm/cc. The cells serve as partitions or compartments of active component vis-a-vis the foam carrier cell matrix. A greater fraction of active material will reside in the voids when the active material is insoluble compared to its solubility in water. Aside from the actives residing in the cells, intermolecular forces between cell walls and moieties comprising the active also may serve to sequester the actives to the cells.

The type of active sequestered, its interaction with the foam carrier protein, and the environment of the body cavity in which the vehicle is placed, will determine whether the delivery system will facilitate a controlled release, a sustained release, or a single rapid release of active ingredient. Choosing the protein type based on its number of associated coordinated complex docking stations to interact with the specific active, and in some cases buffers containing metals, will render the desired release paradigm.

Various mechanical means are utilized to produce the dehydrated edible foam carrier, including, but not limited to whipping, vacuum puffing, or otherwise aerating biological material. The whipping or aeration motion is effected via a standard planetary mixer or a continuous pressure beater. Aeration can be conducted using ambient air at ambient temperature and pressure. Alternatively, aeration can be conducted with a relatively inert gas (such as nitrogen) either pressurized or at ambient temperature. In the case of vacuum puffing, a pull of up to 28 inches of mercury is suitable to effect aeration.

The use of pressurization has a direct impact on the product's characteristics (i.e., cell structure and shape). This in turn has an effect on the carrier's solubility and friability. For example, the use of inert gasses (including, but not limited to, nitrogen, helium, and argon) during pressurization may protect actives from oxidation (and therefore loss of effectiveness) over time.

Pressurization is effected with air, inert gas, or a combination of the two at between 0-120 psi. This pressure range is suitable for both batch and continuous beating. When continuous beating only is utilized, wherein a 0-100 psi back pressure is used, then the preferred pressure is between 40-60 psi.

The creation of an enhanced volume foam carrier is facilitated with the presence of leavening agents and/or effervescent agents in the liquor. Such agents produce vapor which is trapped or otherwise sequestered in the matrix created by the coagulated proteins. This results in foam instability and eventual cell breakdown.

As noted supra, over agitation is to be avoided, inasmuch as over-beaten protein causes unwanted coagulation, which confounds the formation of an otherwise homogenous foam construct. To guard against coagulation, metal chelation, complexation or coordination can be utilized, whereby metal ions complex with the albumen constituent, conalbumin, which confounds and weakens the formation. This complex is more stable than conalbumin alone so that it resists denaturing. Exemplary metal ions utilized in this anti-clumping protocol include, but are not limited to copper, sodium, iron, calcium, potassium and magnesium, and are supplied as cations of such common salts as gluconate, citrate, tartrate, sulfate, phosphate, etc. The concentration of these metals is empirically derived. Any amount of metal is useful up to the amount necessary to bond with all coordination sites of the specific protein employed. Divalent cations provide economy of use inasmuch as they bond and deactivate two coordination sites.

The resulting aerated mass is then extruded or deposited into desired geometric shaped molds, dried and finally packaged in moisture proof containers or wrapping.

An advantage of the invented formulation is that its viscosity allows it to be cut or pressed into desired shapes and sizes while still wet. As a result, a skin develops over the outer, still moist surface of the final construct, resulting in a more homogeneous surface when the construct finally dries. This smooth surface is less prone to crumbling, less prone to localized wicking (i.e. uptake) of ambient fluid during storage and initial dispensing, and less prone to outgassing of sequestered vapor-phase constituents. A longer shelf life and a more controlled release of active material residing within the confines of the smooth construct is therefore assured. Alternate manufacturing methods allow a cellular structure to exist on the top and bottom (and/or the sides) of the construct. These alternate methods include depositing of the formulated liquor into starch molds, or into molds comprised of relatively inert, manmade materials.

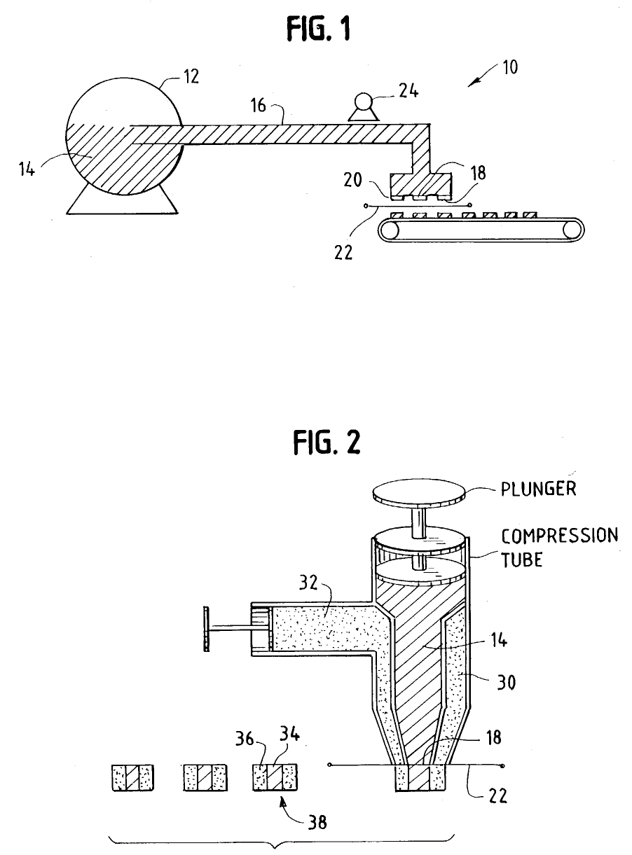

An exemplary protocol for producing discrete foam constructs is depicted as numeral 10 in FIG. 1. As noted supra, a feature of the foam construct is its ability to be formed with generally smooth surfaces. This smooth surface feature is advantageous when medical personnel endeavor to place the active bolus in hard to reach treatment sites within certain cavities before release of active compounds commences.

An agitating/aerating device 12 is first utilized to homogeneously blend the constituents of the foam construct with active (i.e. medicament) material. The homogeneous liquor 14 is in communication with an extrusion port 18 (or a plurality of extrusion ports). This communication is effected via a conduit 16 or some other means of physical transport.

Upon extrusion of the liquor from the extrusion port 18, a means 22 for separating extruded portions 20 of the liquor from its feed is provided. The separation means 22 depicted in FIG. 1 is a simple wire which effects a transverse slice through the downwardly depending extruded portion 20. Other cutting means include, but are not limited to water jet, air jet, ultrasonics, knife with serated or smooth edge or combinations of these.

Generally, cutting or deposition operations occur when the product has a moisture content of approximately 3-5 weight percent.

Extrusion processes require a pressure through the extrusion ports 18. Such pressure can be effected via a pump 24 situated intermediate the aerating device 12 and the extrusion ports 18. Alternatively, the aerating device 12 can impart the pressure necessary to effect transport of the liquor 14 through the extrusion ports 18.

FIG. 2 is a schematic depiction of a co-extrusion process. Generally, a similar aeration device 12 is utilized in this protocol as in the protocol depicted in FIG. 1. However, upstream from the extrusion ports 18, the conduit 16 of the previous protocol is modified to facilitate co-extrusion of a material 32 so as to form a two-phase carrier 38. The two-phase carrier comprises a foam-construct/medicament inner core 34 surrounded by a sleeve 36 of additional material. This additional material is a mixture of the homogeneous liquor 14, but containing less medicament so as to facilitate placement of the carrier in target interstices prior to dispensing of a major dosage of the medicament. Alternatively, the material is a slow dissolving coating.

Coextrusion also allows the center core to be non-aerated and therefore capable of carrying actives normally not compatible with aerated foams, i.e., fatty or oil systems. Generally, the coextrusion process provides for a controlled release or sustained release of medicaments, compared to a single phase, single-extruded or deposited construct. Several extruders are commercially available for use to produce the invented edible foam carrier. For example, the MAKAT Depositor/Extruder, available through Robert Bosch, GmbH and Co. of Viersen, Germany is a suitable instrument. This same device enables the invented process of co-extruding different medicaments and materials to the same construct.

After extrusion and/or deposition, the product is dried to a final moisture of below approximately one percent by weight. Any method (e.g., freeze drying) to effect this final weight is suitable, depending on the heat sensitivity of the medicaments sequestered in the carrier. For example, while oven drying at temperatures of 160 F is typical, temperatures as low as 100 F are suitable.

Optionally, dehumidification can be used to complement drying temperatures. In these instances, drying can occur at room temperatures and low humidity conditions, i.e., below 40 percent humidity.

Alternatively, a continuous belt drying tunnel could be utilized, employing zone heating whereby the initial zone would be warm and humid, a second zone would be hot an drier, and then finally the product is subjected to a hot an dry zone. This zoning effect allows the foam to dry from the inside out, thereby eliminating any surface crusting.

A myriad of medicaments are suitable for delivery via the invented formulation. Exemplary medicaments include, but are not limited to, calcium carbonate, Triamterene, Ticlopidine HCl, Carbamazepine, Astemizole, and others. Generally, medicaments having chemical moieties which are relatively inert with the constituents of the foam construct are suitable.

The following examples present exemplary formulations and weights for the invented foam construct. These examples are merely illustrative and thus are not to be construed as limiting the scope of the invention. For example, the weight percents depicted in the examples are derived empirically, and in some cases after a formulation has been determined to display desired characteristics.

### EXAMPLES

In a first formulation, considered to be albumen based, approximately 200 grams of water is placed in a Hobart-style planetary mixer having a whip attachment. To this is added 100 grams of the albumen-based dry mixture in the following percentages and in the following sequence:

Approximately 9 grams of egg albumen is added to the water and allowed to solubilize, thereby creating a solubilized liquid phase. Solubilization usually occurs in approximately 10 minutes. Solubilization is evident when the liquid has thickened and no residual particles are seen. Proper hydration or solubilization will maintain foam stability and strength.

To the solubilized phase is added approximately 15.5 grams of sugar (a preferred bulking agent), to form a thickened solubilized phase. The aeration (i.e. whipping) process is begun, after which is added approximately 38 grams of confectioner's sugar, 9 grams of TH 1 stabilizer and approximately 28 grams of active ingredient, such as calcium carbonate. (The formula for TH1 stabilizer is approximately 20 weight percent corn starch, 55 weight percent dried egg albumen, 20 weight percent confectioner's sugar, 2 weight percent gum arabic, 0.5 weight percent dicalcium phosphate dihydrate, 1-2 weight percent anhydrous citric acid and 1-2 weight percent sodium bicarbonate.)

This thicken solubilized phase is aerated for a time sufficient to raise the viscosity of the liquor to approximately between 1 to 100,000 centipoises (cp). However, approximately 15 seconds after addition of the confectioner's sugar, stabilizer and active ingredient, aeration should cease, with the components clinging to the side of the mixing vessel scraped into the bulk of the liquor. Homogenization should then commence for a time sufficient to thoroughly mix all ingredients and achieve the desired viscosity.

Upon attainment of the targeted viscosity, the resulting aerated mixture is extruded into desired shapes and subjected to drying at approximately 140° F. until dry.

The above protocol is utilized with the following other formulations:

Second Formulation:

| Constituent | Dry Weight Percent* |
|---|---|
| Dried Egg Albumen | 6 |
| Hydrofoama DSN | 3 |
| Granulated Sugar | 15-16 |
| Confectioner's Sugar | 37-38 |
| Stabilizer TH1 | 9 |
| Calcium Carbonate | 28 |

*Percentages are rounded so that final weight may not be exactly 100.

Third Formulation:

| Constituent | Wet Weight Percent |
|---|---|
| Dried Egg Albumen | 3 |
| Granulated Sugar | 5 |
| Water | 67 |
| Confectioner's Sugar | 12-13 |
| Stabilizer TH1 | 3 |
| Calcium Carbonate | 9-10 |

Fourth Formulation

| Constituent | Wet Weight Percent |
|---|---|
| Dried Egg Albumen | 2 |
| Hyfoama DSN | 1 |
| Granulated Sugar | 5 |
| Water | 67 |
| Confectioner's Sugar | 12-13 |
| Stabilizer | 3 |
| Calcium Carbonate | 9-10 |

Fifth Formulation

| Constituent | Dry Weight Percent |
|---|---|
| Corn Starch | 20 |
| Dried Egg Albumen | 55 |
| Confectioner's Sugar | 20 |
| Gum Arabic | 2 |
| Dicalcium Phosphate Dihydrate | 0.5 |
| Anhydrous Citric Acid | 1-2 |
| Sodium Bicarbonate | 1-2 |
| Vanillin | Trace^{†} |

^{†}Add to taste

The constituents in the fifth formulation are added in a stainless steel mixing vessel and dry blended to homogeniety.

Sixth Formulation

The sixth formulation is an example of a batched and sheeted material suitable for cutting with saw blades or ultrasonic knives. It is an aerated matrix produced chemically by using sodium bicarbonate in a very hot carbohydrate or polyol syrup. The bicarbonate decomposes to give off carbon dioxide, which aerates the syrup.

| Constituent | Dry Weight Percent | |
|---|---|---|
| Water | 4 | lbs. |
| Sugar | 11 | lbs. |
| 43 D.E. Corn Syrup | 10 | lbs. |
| Gelatin 150 Bloom | 2 | oz. |
| Water* | 4 | oz. |
| Sodium Bicarbonate | 12 | oz |

D.E. (Dextrose Equivalent) is an measure of the reducing sugar content of a sweetener, calculated as Dextrose and expressed as a percentage of the total dry substance. As defined in British Standard, B.S. 757, the strength of gelatin is classified according to what is called the bloom strength, which is defined as the force required for a plunger of defined shape and size to make a 4 mm depression in a gel that has been prepared at 6.67% w/w concentration and chilled at 10° C. in a bloom jar for 16-18 hours. The force, recorded in grams, is measured using a texture analyzer. Commercially, gelatins are available with bloom strengths from 50-300 bloom.

The gelatin is soaked in the water and allowed to stand for one hour. Sugar, water and corn syrup are mixed, dissolved and heated to 320 F. Upon attaining that 320 F temperature, the heated mixture is cooled to 270 F and gelatin is added.

The liquor is then removed from heat and placed into insulated sheet molds to stand over night, covered. Mechanical cutters are used to portion the solidified substrate to dosage sizes.

While the invention has been described with reference to details of the illustrated embodiment, these details are not intended to limit the scope of the invention as defined in the appended claims.

The embodiment of the invention in which an exclusive property or privilege is claimed is defined as follows:

1. An edible foam carrier comprising aerated stabilizer with localized cell compartments adapted to receive a medicament.

2. The carrier as recited in claim 1 wherein the stabilizer is egg albumen.

3. The carrier as recited in claim 1 wherein the stabilizer contains metal.

4. The carrier as recited in claim 1 wherein the carrier is adapted to receive the medicament in a medicament:carrier weight ratio of up to approximately 33:100.

5. The carrier as recited in claim 1 wherein the stabilizer is mixed with leavening agents and effervescent agents.

6. The carrier as recited in claim 5 wherein the stabilizer is mixed with carbohydrate.

7. The carrier as recited in claim 1 wherein the carrier weighs from between approximately 0.05 g/cc to 0.9 g/cc.

8. The carrier as recited in 1 wherein the carrier maintains a solid phase at a pH ranging from between approximately 4 and 10.

9. A method for producing an extrudable foam carrier having localized cellular compartments, the method comprising:
a) aerating a water-soluble stabilizer to create a three-dimensional structure, and;
b) confining medicament to the compartments.

10. The method as recited in claim 9, wherein the aerated stabilizer is contacted with leavening agents and effervescence agents.

11. The method as recited in claim 9 wherein the step of confining medicament to the structure further comprises contacting medicament to the structure while the structure is in a state of dehydration.

12. The method as recited in claim 9 wherein the step of confining medicament to the structure further comprises homogeneously mixing the stabilizer with the medicament.

13. The method as recited in claim 9 wherein wet-foam stabilizer is added to the stabilizer prior to aeration.

14. The method as recited in claim 9 wherein the protein is aerated under pressure selected from between 0 and 120 psi

15. The method as recited in claim 9 wherein the medicament is insoluble.

16. The method as recited in claim 10 wherein metal ions are contacted with the protein during leavening.

17. The method as recited in claim 9 wherein the three-dimensional structure is stabilized with the presence of metal ions.

18. The method as recited in claim 17 wherein the metal ions are cations of salts selected from the group consisting of copper, sodium, iron, calcium, potassium, and magnesium.

19. The method as recited in claim 9 wherein confining the medicament to the structure further comprises forming a sleeve over the structure.

20. The method as recited in claim 19 wherein the sleeve does not contain cellular compartments.
